(19) United States
(12) Patent Application Publication
(10) Pub. No.: US 2002/0085997 A1
(43) Pub. Date: Jul. 4, 2002

(54) TUMOUR VACCINE AND PROCESS FOR THE PREPARATION THEREOF

(76) Inventors: WALTER SCHMIDT, VIENNA (AU); MAX BIRNSTIEL, VIENNA (AU); TAMAS SCHWEIGHOFFER, VIENNA (AU); PETER STEINLEIN, VIENNA (AU); MICHAEL BUSCHLE, BRUNN (AU)

Correspondence Address: STERNE KESSLER GOLDSTEIN & FOX, 1100 NEW YORK AVENUE NW, SUITE 600, WASHINGTON, DC, 200053934

(21) Appl. No.: 09/077,214

(22) PCT Filed: Nov. 21, 1996
(86) PCT No.: PCT/EP96/05126

(30) Foreign Application Priority Data
Nov. 23, 1995 (DE) P195 43 649.0
Feb. 24, 1995 (DE) P196 07 044.9

(51) Int. Cl. A61K 48/00; A01N 63/00; A01N 65/00; A61K 39/00; C12N 5/02

(52) U.S. Cl. 424/93.21; 424/93.7; 424/277.1; 435/325

(57) ABSTRACT

Tumor vaccine and process for the preparation thereof. The tumor vaccine contains tumor cells at least some of which contain at least one MHC-I-haplotype of the patient on the cell surface, and which are charged with one or more peptides binding to the MHC-I molecule in such a way that the tumor cells are recognized as foreign by the patient's immune system in context with the peptides and trigger a cellular immune response. The charging is carried out in the presence of a polycation such as polylysine.

The development of a therapeutic vaccine based on tumour cells is essentially dependent on the following conditions: there are qualitative or quantitative differences between tumour cells and normal cells; the immune system is fundamentally capable of recognising these differences; the immune system can be stimulated—by active specific immunisation with vaccines—to recognise tumour cells by means of these differences and cause them to be rejected.

In order to achieve an anti-tumour response, at least two conditions must be satisfied: firstly, the tumour cells must express antigens or neo-epitopes which do not occur on normal cells. Secondly, the immune system must be activated accordingly in order to react to these antigens. A serious obstacle in the immune therapy of tumours is their low immunogenicity, particularly in humans. This is surprising in as much as one might expect the large number of genetic changes in malignant cells to lead to the formation of peptide neo-epitopes, which can be recognised in context with MHC-I-molecules of cytotoxic T-lymphocytes.

Recently, tumour-associated and tumour-specific antigens have been discovered which constitute such neo-epitopes and thus ought to constitute potential targets for an attack by the immune system. The fact that the immune system nevertheless does not succeed in eliminating the tumours which express these neo-epitopes would then obviously not be due to the absence of neo-epitopes but due to the fact that the immunological response to these neo-antigens is inadequate.

For immunotherapy of cancer on a cellular basis, two general strategies have been developed: on the one hand, adoptive immunotherapy which makes use of the in vitro expansion of tumour-reactive T-lymphocytes and their reintroduction into the patient; on the other hand, active immunotherapy which uses tumour cells in the expectation that this will give rise to either new or more powerful immune responses to tumour antigens, leading to a systemic tumour response.

Tumour vaccines based on active immunotherapy have been prepared in various ways; one example consists of irradiated tumour cells mixed with immunostimulant adjuvants such as Corynebacterium parvum or Bacillus Calmette Guerin (BCG) in order to provoke immune reactions against tumour antigens (Oettgen and Old, 1991).

In recent years, in particular, genetically modified tumour cells have been used for active immunotherapy against cancer, the foreign genes introduced into the tumour cells falling into three categories:

One of these uses tumour cells which are genetically modified in order to produce cytokines. The local coincidence of tumour cells and cytokine signal are supposed to provide a stimulus which triggers the anti-tumour immunity. A survey of applications of this strategy is provided by Pardoll, 1993, Zatloukal et al., 1993, and Dranoff and Mulligan, 1995.

Tumour cells which have been genetically modified in order to secrete cytokines such as IL-2, GM-CSF or IFN-γor in order to express co-stimulating molecules have been shown, in experimental animal models, to generate potent anti-tumour immunity (Dranoff et al., 1993; Zatloukal et al., 1995). However, in a human being who already has a substantial tumour and has developed a tolerance to the tumour, it is substantially more difficult to detect the cascade of complex interactions completely in order that an effective anti-tumour reaction can take place. The actual effectiveness of cytokine-secreting tumour vaccines for use in humans has not yet been demonstrated.

Another category of genes with which tumour cells have been modified for use as tumour vaccines codes for so-called accessory proteins; the objective of this approach is to convert tumour cells into antigen-presenting cells (neo-APCs) in order to allow them to generate tumour-specific T-lymphocytes directly. An example of an approach of this kind is described by Ostrand-Rosenberg, 1994.

The identification and isolation of tumour antigens (TAs) or peptides derived therefrom, e.g. as described by Wölfel et al., 1994 a) and 1994 b); Carrel et al., 1993, Lehmann et al., 1989, Tibbets et al., 1993, or in the published International Applications WO 92/20356, WO 94/05304, WO 94/23031, WO 95/00159) was the prerequisite for using tumour antigens as immunogens for tumour vaccines, both in the form of proteins and in the form of peptides. However, a tumour vaccine in the form of tumour antigens as such is not sufficiently immunogenic to trigger a cellular immune response which would be necessary to eliminate tumour cells carrying tumour antigen; the co-administration of adjuvants provides only limited possibilities for intensifying the immune response (Oettgen and Old, 1991).

A third strategy for active immunotherapy in order to increase the efficacy of tumour vaccines is based on xenogenised (alienised) autologous tumour cells. This concept is based on the assumption that the immune system reacts to tumour cells which express a foreign protein and that, in the course of this reaction, an immune response is also provoked against those tumour antigens (TAs) which are presented by the tumour cells of the vaccine.

A summary of these various approaches in which tumour cells are alienised for the purpose of greater immunogenicity by the introduction of various genes is given by Zatloukal et al., 1993.

A central role is played in the regulation of the specific immune response by a trimolecular complex consisting of the components of T-cell-antigen receptor, MHC (Major Histocompatibility Complex) molecule and the ligand thereof which is a peptide fragment derived from a protein.

MHC-I molecules (or the corresponding human molecules, the HLAs) are peptide receptors which allow the binding of millions of different ligands, with stringent specificity. The prerequisite for this is provided by allele-specific peptide motifs which have the following specificity criteria: the peptides have a defined length, depending on the MHC-I haplotype, this length generally being from eight to ten amino acid groups. Typically, two of the amino acid positions are so-called “anchors” which can only be occupied by a single amino acid or by amino acid groups with closely related side chains. The exact position of the anchor amino acids in the peptide and the requirements made on their properties vary with the MHC-I-haplotypes. The C-terminus of the peptide ligands is frequently an aliphatic or a charged group. Such allele-specific MHC-I-peptide-ligand motifs have hitherto been known, inter alia, for H-2K^{d}, K^{b}, K^{k}, K^{km1}, D^{b}, HLA-A*0201, A*0205 and B*2705.

Within the scope of the protein conversion inside the cell, regular, degenerate and foreign gene products, e.g. viral proteins or tumour antigens, are broken down into small peptides; some of them constitute potential ligands for MHC-I molecules. This provides the prerequisite for their presentation by MHC-molecules and, as a result, the triggering of a cellular immune response, although it has not yet been clearly explained how the peptides are produced as MHC-I ligands in the cell.

One approach which makes use of this mechanism for the alienisation of tumour cells in order to intensify the immune response consists in treating tumour cells with mutagenic chemicals such as N-methyl-N′-nitroso-guanidine. This is supposed to cause the tumour cells to present neo-antigens derived from mutated variants of cellular proteins, constituting foreign gene products (Van Pel and Boon, 1982). However, since the mutagenic events are randomly distributed over the genome and additionally some cells can be expected to present different neo-antigens as a result of different mutagenic events, this process is difficult to control from a qualitative and quantitative point of view.

Another approach alienises tumour cells by transfecting them with genes of one or more foreign proteins, e.g. that of a foreign MHC-I molecule or MHC proteins of different haplotypes, which then appears in form on the cell surface (EP-A2 0 569 678; Plautz et al., 1993; Nabel et al., 1993). This approach is based on the idea mentioned above that the tumour cells, when administered in the form of a whole cell vaccine, are recognised as foreign by means of the expressed protein or the peptides derived therefrom, or that, in the event of the expression of autologous MHC-I molecules, the presentation of tumour antigen is optimised by an increased number of MHC-I molecules on the cell surface. The modification of tumour cells with a foreign protein may cause the cells to present peptides originating from the foreign protein in the MHC context and the modification from “self” to “foreign” takes place within the scope of the MHC-peptide complex recognition. The recognition of a protein or peptide as being foreign means that, in the course of the immune recognition, an immune response is produced not only against the foreign protein, but also against the tumour antigens belonging to the tumour cells. In the course of this process, the antigen-presenting cells (APCs) are activated; they process the proteins (including TAs) occurring in the tumour cell of the vaccine to form peptides and use them as ligands for their own MHC-I and MHC-II molecules. The activated, peptide-charged APCs migrate into the lymph nodes, where a few of the immature T-lymphocytes recognise the peptides originating from the TA on the APCs and are able to use them as a stimulus for clonal expansion - in other words in order to generate tumour-specific CTLs and T-helper cells.

The aim of the present invention is to provide a new tumour vaccine based on alienised tumour cells, by means of which an effective cellular anti-tumour immune response can be initiated.

In solving this problem, the following considerations were taken as basic premises: whereas non-malignant normal body cells are tolerated by the immune system, the body reacts to a normal cell by means of an immune response if this cell synthesises proteins foreign to the body, e.g. as the result of a viral infection. The reason for this is that the MHC-I molecules present foreign peptides which originate from the foreign proteins. Consequently, the immune system registers that something undesirable and alien has happened to this cell. The cell is eliminated, APCs are activated and a new specific immunity is generated against the cells expressing the foreign proteins.

Tumour cells admittedly contain the tumour-specific tumour antigens in question but are ineffective vaccines as such, because they are ignored by the immune system as the result of their low immunogenicity. If, by contrast to the known approaches, a tumour cell were to be charged not with a foreign protein but with a foreign peptide, in addition to the foreign peptides the cell's own tumour antigens will be recognised as foreign by this cell. By alienisation with a peptide the intention is to direct the cellular immune response triggered by the foreign peptides against the tumour antigens.

The reason for the low immunogenicity of tumour cells may not be a qualitative problem but a quantitative problem. For a peptide derived from a tumour antigen, this may mean that it is indeed presented by MHC-I molecules but in a concentration which is too low to trigger a cellular tumour-specific immune response. An increase in the number of tumour-specific peptides on the tumour cell should thus also result in alienisation of the tumour cell, resulting in the triggering of a cellular immune response. In contrast to approaches in which the tumour antigen or the peptide derived from it is presented on the cell surface by the fact that it has been transfected with a DNA coding for the protein or peptide in question, as described in International Publications WO 92/20356, WO 94/05304, WO 94/23031 and WO 95/00159, the intention is to provide a vaccine which triggers an efficient immune response whilst being simpler to manufacture.

Mandelboim et al., 1994 and 1995 propose that RMA-S cells be incubated with peptides derived from tumour antigens in order to trigger a cellular immune response against the corresponding tumour antigens native to the patient. The cells known as RMA-S (Kärre et al., 1986) proposed for tumour vaccination by Mandelboim et al. are assumed to be able to act as APCs. They have the peculiarity that their HLA molecules on the cell surface are empty as the result of a defect in the cellular TAP mechanism (transport of antigenic peptides; responsible for the processing of peptides and their binding to HLA molecules). Consequently, the cells are available for charging with a peptide and thus simultaneously function as a presenting vehicle for the peptide provided from outside. The anti-tumour effect achieved is based on triggering an immune response to the peptide presented on the cells, which is offered to the immune system without any direct context with the antigenic repertoire of the tumour cell.

The invention relates to a tumour vaccine for administering to a patient, consisting of tumour cells which themselves present peptides derived from tumour antigens in the HLA context and at least some of which have at least one MHC-I-haplotype of the patient on the cell surface and which are charged with one or more peptides a) and/or b) in such a way that the tumour cells are recognised as foreign in context with the peptides of the patient's immune system and trigger a cellular immune response, these peptides

a) acting as ligands for the MHC-I-haplotype, which is common to the patient and to the tumour cells in the vaccine, and are different from peptides derived from proteins which are expressed by the patient's cells, or

b) acting as ligands for the MHC-I-haplotype which is common to the patient and the tumour cells of the vaccine, and are derived from tumour antigens expressed by the patient's cells and occur in a concentration on the tumour cells of the vaccine which is higher than the concentration of a peptide derived from the same tumour antigen as the one expressed on the patient's tumour cells.

The human MHC molecules are hereinafter also referred to as HLA (Human Leucocyte Antigen) in accordance with International Conventions.

The term “cellular immune response” denotes the cytotoxic T-cell immunity which, as a result of the generation of tumour-specific cytotoxic CD8-positive T-cells and CD4-positive helper-T-cells, brings about destruction of the tumour cells.

The effectiveness of the vaccines according to the invention obtained from tumour cells is based primarily on the fact that the immunogenic activity of the supply of tumour antigens present on the tumour cells is intensified by the peptide.

The peptides of type a) are hereinafter also referred to as “foreign peptides” or “xenopeptides”.

In one embodiment of the invention, the tumour cells of the vaccine are autologous. These are cells taken from the patient who is to be treated, the cells are treated ex vivo with peptide or peptides a) and/or b), optionally inactivated and then re-administered to the patient. (Methods for producing autologous tumour vaccines are described in WO 94/21808, the contents of which are hereby referred to).

In one embodiment of the invention, the tumour cells are allogenic, i.e. they do not come from the patient being treated. The use of allogenic cells is particularly preferred when economic considerations are involved; the production of individual vaccines for each individual patient is labour-intensive and expensive and moreover, problems occur in individual patients in the ex vivo cultivation of the tumour cells, with the result that tumour cells are not obtained in sufficiently large numbers for the preparation of a vaccine. With the allogenic tumour cells, it should be borne in mind that they have to be matched to the HLA-subtype of the patient.

When foreign peptides of category a) are used, in the case of allogenic tumour cells, these are cells of one or more cell lines, of which at least one cell line expresses at least one and preferably more tumour antigens which are identical to the tumour antigens of the patient to be treated, i.e. the tumour vaccine is matched to the tumour indication of the patient. This ensures that the cellular immune response triggered by the MHC-I-presenting foreign peptides to the tumour cells of the vaccine, leading to the expansion of tumour-specific CTLs and T-helper cells, is also directed against the tumour cells in the patient, as they express the same tumour antigen as the cells of the vaccine.

If, for example, the tumour vaccine according to the invention is to be used to treat a patient suffering from breast cancer metastases which show an Her2/neu-mutation (Allred et al., 1992; Peopoles et al., 1994; Yoshino et al., 1994 a); Stein et al., 1994; Yoshino et al., 1994 b); Fisk et al., 1995; Han et al., 1995) the vaccine used will consist of allogenic tumour cells matched to the HLA-haplotype of the patient, which also express the mutated Her2/neu as tumour antigen. Recently, numerous tumour antigens have been isolated and their connection with one or more cancers have been clarified. Other examples of such tumour antigens are ras (Fenton et al., 1993; Gedde Dahl et al., 1992; Jung et al., 1991; Morishita et al., 1993; Peace et al., 1991; Skipper et al., 1993) MAGE-tumour antigens (Boon et al., 1994; Slingluff et al., 1994; van der Bruggen et al., 1994; WO 92/20356); a survey of various tumour antigens is also provided by Carrel et al., 1993.

A summary of known tumour antigens which may be used within the scope of the invention and peptides derived therefrom is given in the Table.

The tumour antigens of the patient are generally determined in the course of drawing up the diagnosis and treatment plan by standard methods, e.g. using assays based on CTLs with specificity for the tumour antigen which is to be detected. These assays have been described, for example, by Hérin et al., 1987; Coulie et al., 1993; Cox et al., 1994; Rivoltini et al., 1995; Kawakami et al., 1995; and have been described in WO 94/14459; these references also disclose various tumour antigens and peptide epitopes derived therefrom. Tumour antigens occurring on the cell surface can also be detected by immunoassays based on antibodies. If the tumour antigens are enzymes, e.g. tyrosinases, they can be detected using enzyme assays.

In another embodiment of the invention, a mixture of autologous and allogenic tumour cells can be used as the starting material for the vaccine. This embodiment of the invention is used particularly when the tumour antigens expressed by the patient are unknown or only partly characterised and/or when the allogenic tumour cells express only some of the tumour antigens of the patient. By adding autologous tumour cells treated with the foreign peptide it is possible to ensure that at least some of the tumour cells in the vaccine contain the maximum possible number of tumour antigen native to the patient. The allogenic tumour cells are those which match the patient in one or more MHC-I-haplotypes.

The peptides of type a) and b) are defined in accordance with the requirement to bind to an MHC-I-molecule, in terms of their sequence, by the HLA subtype of the patient to whom the vaccine is to be given. Determining the HLA-subtype of the patient thus constitutes one of the most important prerequisites for the choice or design of a suitable peptide.

When the tumour vaccines according to the invention are used in the form of autologous tumour cells, the HLA-subtype is automatically obtained as a result of the specificity of the HLA molecule which is genetically determined in the patient. The HLA subtype of the patient can be detected using standard methods such as the micro-lymphotoxicity test (MLC test, Mixed Lymphocyte Culture) (Practical Immunol., 1989). The MLC test is based on the principle of mixing lymphocytes isolated from the patient's blood first with antiserum or a monoclonal antibody against a specific HLA molecule in the presence of rabbit complement (C). Positive cells are lysed and absorb an indicator dye, whereas undamaged cells remain unstained.

RT-PCR can also be used to determine the HLA-haplotype of a patient (Curr. Prot. Mol. Biol. Chapters 2 and 15). In order to do this, blood is taken from the patient and RNA is isolated from it. This RNA is subjected first to reverse transcription, resulting in the formation of cDNA from the patient. The cDNA is used as a matrix for the polymerase chain reaction with primer pairs which specifically bring about the amplification of a DNA fragment which represents a certain HLA-haplotype. If after agarose gel electrophoresis a DNA band appears, the patient expresses the corresponding HLA molecule. If the band does not appear, the patient is negative for it. At least two bands can be expected for each patient.

When the invention is applied in the form of an allogenic vaccine, cells are used of which at least some are matched to at least one HLA-subtype of the patient. For the purpose of achieving the widest possible application for the vaccines according to the invention, a mixture of different cell lines is preferably used as starting material, expressing two or three different ones of the HLA-subtypes most frequently found, and taking particular account of haplotypes HLA-A1 and HLA-A2. Using a vaccine based on a mixture of allogenic tumour cells which express these haplotypes, it is possible to screen a large population of patients; in this way about 70% of the population of Europe can be covered (Machiewicz et al., 1995).

The definition of the peptides used according to the invention by means of the HLA-subtype defines them in terms of their anchor amino acids and their length; defined anchor positions and length ensure that the peptides fit into the peptide binding fork of the HLA molecule in question and are presented on the cell surface of the tumour cells which form the vaccine in such a way that the cells are recognised as foreign. This means that the immune system will be stimulated and a cellular immune reaction will be provoked against the tumours cells of the patient.

Peptides which are suitable as foreign peptides of category a) for the purposes of the present invention are available in a wide range. Their sequence may be derived from naturally occurring immunogenic proteins or the cellular breakdown product thereof, e.g. viral or bacterial peptides, or from tumour antigens foreign to the patient.

Suitable foreign peptides may be selected, for example, on the basis of peptide sequences known from the literature; e.g. by means of the peptides described by Rammensee et al., 1993, Falk et al., 1991, for the different HLA motifs, peptides derived from immunogenic proteins of various origins, which fit into the binding sites of the molecules of the various HLA-subtypes. For peptides which have a partial sequence of a protein with an immunogenic activity, it is possible to establish which peptides are suitable candidates by means of the polypeptide sequences already known or possibly still to be established, by sequence comparison taking account of the HLA-specific requirements. Examples of suitable peptides are found, for example, in Rammensee et al., 1993, Falk et al., 1991, and Rammensee, 1995 and in WO 91/09869 (HIV peptides); peptides derived from tumour antigens are described, inter alia, in the published International Patent Applications WO 95/00159 and WO 94/05304. Reference is hereby made to the disclosure of these references and the Articles cited therein in connection with peptides.

Preferred candidates for xenopeptides are the peptides whose immunogenicity has already been demonstrated, i.e. peptides derived from known immunogens such as viral or bacterial proteins. Peptides of this kind exhibit a violent reaction in the MLC test on account of their immunogenicity.

Instead of using the original peptides, i.e. peptides which are derived unchanged from natural proteins, it is possible to carry out variations as required, using the minimum requirements regarding anchor positions and lengths, specified on the basis of the original peptide sequence; in this case, therefore, synthetic peptides are used according to the invention which are designed in accordance with the requirements relating to an MHC-I ligand. Thus, for example, starting from the H2-K^{d}-ligand Leu Phe Glu Ala Ile Glu Gly Phe Ile (LFEAIEGFI) it is possible to change the amino acids which are not anchor amino acids in such a way as to obtain the peptide of the sequence Phe Phe Ile Gly Ala Leu Glu Glu Ile (FFIGALEEI); moreover, the anchor amino acid Ile at position 9 can be replaced by Leu.

Peptides derived from tumour antigens, i.e. from proteins which are expressed in a tumour cell and which do not appear in the corresponding untransformed cell or appear only in a significantly lower concentration, may be used within the scope of the present invention as peptides of type a) and/or type b).

The length of the peptide preferably corresponds to the minimum sequence of 8 to 10 amino acids required for binding to the MHC-I molecule, together with the necessary anchor amino acids. If desired, the peptide may also be lengthened at the C- and/or N-terminus provided that this lengthening does not interfere with the binding capacity, i.e. that the extended peptide can be processed at cellular level down to the minimum sequence.

In one embodiment of the invention the peptide may be extended with negatively charged amino acids, or negatively charged amino acids may be incorporated in the peptide, at positions other than the anchor amino acids, in order to achieve electrostatic binding of the peptide to a polycation such as polylysine.

The term “peptides” for the purposes of the present invention by definition includes larger protein fragments or whole proteins which are guaranteed, after application of the APCs, to be processed into peptides which fit the MHC molecule.

In this embodiment, the antigen is thus used not in the form of a peptide but as a protein or protein fragment or as a mixture of proteins or protein fragments. The protein constitutes an antigen or tumour antigen from which the fragments obtained after processing are derived. The proteins or protein fragments received by the cells are processed and can then be presented to the immune effector cells in the MHC context and thus trigger or intensify an immune response (Braciale and Braciale, 1991; Kovacsovics Bankowski and Rock, 1995; York and Rock, 1996).

When proteins or protein fragments are used, the identity of the processed end product can be demonstrated by chemical analysis (Edman degradation or mass spectrometry of processed fragments; cf. the survey by Rammensee et al., 1995 and the origin literature cited therein) or by biological assays (the ability of APCs to stimulate T-cells which are specific to the processed fragments).

In principle, peptide candidates are selected for their suitability as foreign peptides in several stages: generally, the candidates are first tested in a peptide binding test for their binding capacity to an MHC-I molecule, preferably by series of tests.

One suitable method of investigation is, for example, the FACS analysis based on flow cytometry (Flow Cytometry, 1989; FACS Vantage TM User's Guide, 1994; CELL Quest™ User's Guide, 1994). The peptide is marked with a fluorescent dye, e.g. with FITC (fluorescein isothiocyanate) and applied to tumour cells which express the MHC-I molecule. In the flow, individual cells are excited by a laser of a certain wavelength; the fluorescence emitted is measured and is dependent on the quantity of peptide bound to the cell.

Another method of determining the quantity of peptide bound is the Scatchard blot. Peptide labelled with I¹²⁵ or with rare earth metal ions (e.g. europium) is used for this. The cells are charged at 4° C. with various defined concentrations of peptide for 30 to 240 minutes. In order to determine non-specific interaction of peptide with cells, an excess of unlabelled peptide is added to some of the samples, preventing the specific interaction of the labelled peptide. Then the cells are washed to remove any non-specific cell-associated material. The quantity of cell-bound peptide is then determined either in a scintillation counter using the radioactivity emitted, or in a photometer which is suitable for measuring long-lived fluorescence. The data thus obtained are evaluated using standard methods.

In a second step, candidates with good binding qualities are tested for their immunogenicity.

The immunogenicity of xenopeptides derived from proteins the immunogenic activity of which is unknown may be tested, for example, by the MLC test. Peptides which provoke a particularly violent reaction in this test, which is preferably also carried out in series with different peptides, using as standard a peptide with a known immunogenic activity, are suitable for the purposes of the present invention.

Another possible way of testing MHC-I-binding peptide candidates for their immunogenicity consists in investigating the binding of the peptides to T2 cells. One such test is based on the peculiar nature of T2 cells (Alexander et al., 1989) or RMA-S-cells (Kärre et al., 1986) that they are defective in the TAP peptide transporting mechanism and only present stable MHC-I molecules when they are applied to peptides which are presented in the MHC-I context. T2 cells or RMA-S cells stably transfected with an HLA gene, e.g. with HLA-A1 and/or HLA-A2 genes, are used for the test. If the cells are acted upon by peptides which are good MHC-I ligands, by being presented in the MHC-I context in such a way as to be recognised as foreign by the immune system, these peptides cause the HLA molecules to appear in significant quantities on the cell surface. Detection of the HLAs on the cell surface, e.g. by means of monoclonal antibodies, makes it possible to identify suitable peptides (Malnati et al., 1995; Sykulev et al., 1994). Here again, a standard peptide known to have a good HLA- or MHC-binding capacity is appropriately used.

In one embodiment of the invention, an autologous or allogenic tumour cell of the vaccine may have a number of xenopeptides with different sequences. In this case, the peptides used may differ from one another, on the one hand, in that they bind to different HLA subtypes. In this way, it is possible to detect several or all the HLA subtypes of a patient or of a larger group of patients. The vaccine is administered in irradiated form.

Another, possibly additional, variability with regard to the xenopeptides presented on the tumour cell may consist in the fact that peptides which bind to a certain HLA subtype differ in their sequence which is not crucial to HLA binding, being derived, for example, from proteins of different origins, e.g. from viral and/or bacterial proteins. Variability of this kind, which offers the vaccinated organism a wider range of alienisation, can be expected to intensify the stimulation of the immune response.

In the embodiment of the invention in which the tumour vaccine consists of a mixture of allogenic tumour cells of various cell lines and, possibly, additionally autologous tumour cells, all the tumour cells may have been treated with the same peptide or peptides or the tumour cells of different origins may also have different xenopeptides.

In the experiments carried out within the scope of the present invention, a viral peptide of the sequence Leu Phe Glu Ala Ile Glu Gly Phe Ile which is derived from the influenza virus haemagglutinin and is an H2-K^{d}-ligand was used as the foreign peptide of type a); the anchor amino acids are underlined.

A tumour vaccine was produced with this naturally occurring viral peptide as the foreign peptide and it was tested on an animal model (melanoma model and colon carcinoma model).

Another viral peptide of the sequence Ala Ser Asn Glu Asn Met Glu Thr Met, which is derived from the nucleoprotein of the influenza virus and is a ligand of the HLA-1-haplotype H2-K^{b} (Rammensee et al., 1993; the anchor amino acids are underlined) was used to produce a tumour vaccine; the protective effect of the vaccine was confirmed in another melanoma model.

Another vaccine was produced by alienising tumour cells with a foreign peptide of the sequence Phe Phe Ile Gly Ala Leu Glu Glu Ile (FFIGALEEI). This is a synthetic peptide which has not hitherto been found in nature. When choosing the sequence, care was taken to satisfy the requirements regarding the suitability as a ligand for the MHC-I molecule of type H2-Kd. The suitability of the peptide for producing an anti-tumour immunity according to the concept of active immunotherapy was confirmed on a murine colon carcinoma CT-26 (syngenic for the mouse strain Balb/c).

In another embodiment of the invention the tumour vaccine may also contain autologous and/or allogenic tumour cells and/or fibroblasts transfected with cytokine genes. WO 94/21808 and Schmidt et al., 1995 (to which reference is made) describe efficient tumour vaccines produced by means of the DNA transport method known as “transferrinfection” with an IL-2 expression vector (this method is based on receptor-mediated endocytosis and uses a cellular ligand, particularly transferrin, conjugated with a polycation such as polylysine, for complexing DNA, and an endosomolytically active agent such as adenovirus).

Preferably, the peptide-treated tumour cells and the cytokine-expressing cells are mixed in the ratio 1:1. If, for example, an IL-2 vaccine which produces 4,000 units of IL-2 per 1×10⁶ cells is mixed with 1×10⁶ peptide-treated tumour cells, the vaccine thus obtained can be used for two treatments, assuming an optimum dosage of 1,000 to 2,000 units of IL-2 (Schmidt et al., 1995).

By combining the cytokine vaccine with the peptide-treated tumour cells it is advantageously possible to combine the effects of these two types of vaccine.

The working up of the cells and the formulation of the vaccine according to the invention are carried out in conventional manner, as described for example in Biologic Therapy of Cancer, 1991, or in WO 94/21808.

According to another aspect, the invention relates to a process for producing a tumour vaccine consisting of tumour cells for administering to a patient.

The process is characterised according to the invention in that tumour cells which themselves present peptides derived from tumour antigens in an HLA context and of which at least some express at least one MHC-I-haplotype of the patient are treated with one or more peptides which

a) act as ligands for the MHC-I-haplotype which is common to the patient and the tumour cells of the vaccine, and are different from peptides derived from proteins expressed by cells of the patient, or which

b) act as ligands for the MHC-I-haplotype which is common to the patient and the tumour cells of the vaccine, and are derived from tumour antigens expressed by the patient's cells,

the tumour cells being incubated with one or more peptides a) and/or b) for such a time and in such an amount in the presence of an organic polycation that the peptides are bound to the tumour cells in such a way as to be recognised as foreign by the immune system of the patient, in context with the tumour cells, and trigger a cellular immune response.

The quantity of peptide is preferably about 50 μg to about 160 μg per 1×10⁵ up to 2×10⁷ cells. If a peptide of category b) is used the concentration may also be higher. For these peptides it is essential that their concentration on the tumour cells of the vaccine should be higher than the concentration of a peptide on the tumour cells of the patient, derived from the same tumour antigen, to the extent that the tumour cells of the vaccine are recognised as foreign and provoke a cellular immune response.

Suitable polycations include homologous organic polycations such as polylysine, polyarginine, polyornithine or heterologous polycations having two or more different positively charged amino acids, whilst these polycations may have different chain lengths, as well as non-peptidic synthetic polycations such as polyethyleneimines, natural DNA-binding proteins of a polycationic nature such as histones or protamines or analogues or fragments thereof, and spermine or spermidines. Organic polycations which are suitable for the purposes of the present invention also include polycationic lipids (Felgner et al., 1994; Loeffler et al., 1993; Remy et al., 1994; Behr, 1994) which are commercially obtainable, inter alia, as transfectam, lipofectamine or lipofectin.

Polylysine (pL) with a chain length of approximately 30 to 300 lysine groups is preferably used as the polycation.

The quantity of polycation required in relation to the peptide can be determined empirically. If polylysine and xenopeptides of category a) are used, the mass ratio of pL:peptide is preferably about 1:4 to about 1:12.

The incubation period is generally from 30 minutes to 4 hours. It depends on the time when the maximum charge of peptide is reached; the degree of charging can be monitored by FACS analysis and in this way the necessary incubation period can be determined.

In another embodiment of the invention, the polylysine is used in an at least partially conjugated form. Preferably, some of the polylysine is in a form conjugated with transferrin (Tf) (namely transferrin-polylysine conjugate TfpL, for which reference is made to the disclosure of WO 94/21808), the mass ratio of pL:TfpL preferably being about 1:1.

Instead of being conjugated with transferrin, polylysine may also be conjugated with other proteins, e.g. the cellular ligands described as internalising factors in WO 94/21808.

Treatment of the tumour cells may also, if desired, be carried out in the presence of DNA. The DNA is preferably in the form of a plasmid, preferably a plasmid which is free from sequences coding for functional eukaryotic proteins, i.e. in the form of an empty vector. In theory, any current, functionally obtainable plasmid may be used as the DNA.

The quantity of DNA in relation to the polycation which is optionally partly conjugated with a protein, e.g. in relation to pL, TfpL or a mixture of pL and TfpL, is preferably about 1:2 to about 1:5.

The incubation period, the quantity and nature of the polycation in relation to the number of tumour cells and/or the amount of peptide, the question whether and in what proportions the polycation is conjugated or with which protein it is best conjugated, the advantage of the presence of DNA and the amount thereof may all be determined empirically. In order to do this, the individual parameters of the process are varied and the peptides are applied to the tumour cells under otherwise identical conditions and examined to see how efficiently the peptides have bound to the tumour cells. One suitable method of doing this is FACS analysis.

The process according to the invention is suitable not only for treating tumour cells but also for treating other cells.

Instead of tumour cells, autologous fibroblasts, i.e. those native to the patient, or cells from fibroblast cell lines which are either matched to the HLA-subtype of the patient or have been transfected with the corresponding MHC-I gene, may be charged by the process according to the invention with one or more peptides derived from tumour antigens expressed by the tumour cells of the patient. The fibroblasts thus treated and irradiated may be used as they are or mixed with peptide-treated tumour cells as a tumour vaccine.

In another embodiment, instead of fibroblasts, dendritic cells may be treated by the process according to the invention. Dendritic cells are APCs of the skin; they may be charged in vitro, as required, i.e. cells isolated from the patient are mixed in vitro with one or more peptides, the peptides being derived from tumour antigens of the patient and binding to an MHC-I or an MHC-II molecule of the patient. In another embodiment, these cells may also be charged with the peptide in vivo. In order to do this, the complexes of peptide, polycation and optionally DNA are preferably injected intradermally, as dendritic cells are particularly frequently found in the skin.

Within the scope of the present invention, the peptide was complexed with TfpL or pL for transfer into CT-26 cells and with TfpL and a non-functional plasmid (empty vector) for transfer into M-3 cells. In the CT-26 system it was found that the irradiated tumour vaccines alienised with the peptide generated an efficient anti-tumour immunity: 75% of the vaccinated mice were able to eliminate a tumour challenge which resulted in tumour formation in all the control animals, which were either given no vaccine or were given a vaccine without the xenopeptide. In the M-3 system, the same xenopeptide was tested in an experimental set-up adapted to the situation in humans, under conditions which are even more stringent for the organism in terms of tumour formation. Mice with metastases were vaccinated with xenopeptised irradiated M-3 cells. 87.5% of the mice thus vaccinated were able to eliminate the metastases, whilst all the untreated mice and 7/8 mice who had been given the vaccine without the xenopeptide fell ill with tumours.

It was also found that the degree of systemic immune response of the tumour vaccines depends on the method by which the peptide is applied to the tumour cells. When the peptide was administered to the cells by polylysine/transferrin, the effect was significantly more marked than when the cells were incubated with the peptide for 24 hours (“pulsing”). The adjuvant mixing of the peptide with the irradiated vaccines was also inefficient. The transferrinfection would appear to have either ensured more efficient uptake of the peptide in the cells or the charging with polylysine/transferrin would appear to cause the peptide to remain stuck on the cell membrane and thus be brought physically close to the MHC-I molecule and then be able to bind to it, with the possibility of its displacing cellular peptides which are weakly bound owing to its strong affinity.

### SUMMARY OF FIGURES

FIG. 1a-c: FACS-analysis of M-3 cells treated with foreign peptide

FIG. 1d: Microphotographs of M-3 cells treated with FITC peptide

FIGS. 2a,b: Curing of DBA/2 mice having M-3 melanoma metastases, using a vaccine of M-3 cells charged with foreign peptide

FIG. 3a: Titration of foreign peptide for the production of a tumour vaccine

FIG. 3b: Comparison of a tumour vaccine of tumour cells charged with foreign peptide, with a tumour vaccine secreting IL-2

FIG. 4a: Protection of Balb/c mice by pre-immunisation with a vaccine from colon carcinoma cells charged with foreign peptide

FIG. 4b: Investigation of the participation of T-cells in systemic immunity

FIG. 5: Protection of C57BL/6J mice by pre-immunisatiion with a vaccine of melanoma cells charged with foreign peptide

In the Examples which follow, the following materials and methods were used unless otherwise stated:

The murine melanoma cell line Cloudman S91 (clone M-3; ATCC No. CCL 53.1) was obtained from ATCC.

The melanoma cell line B16-F10 (Fidler et al., 1975) was obtained from the NIH DCT tumour depository.

The preparation of transferrin-polylysine-conjugates from transfection complexes containing DNA was carried out as described in WO 94/21808.

The peptides LFEAIEGFI, FFIGALEEI, LPEAIEGFG and ASNENMETM were synthesised in a peptide synthesiser (Model 433 A with feedback monitor, Applied Biosystems, Foster City, Canada) using TentaGel S PHB (Rapp, Tübingen) as a solid phase using the Fmoc method (HBTU activation, Fastmoc™, scale 0:25 mmol). The peptides were dissolved in 1 M TEAA, pH 7.3, and purified by reverse chromatography on a Vydac C 18 column. The sequences were confirmed by flight time mass spectrometry on an MAT Lasermat (Finnigan, San Jose, Canada).

Testing the effectiveness of the cancer vaccines for their protective effect against metastasis formation (“therapeutic mouse model”) and testing in the prophylactic mouse model were carried out using the procedure described in WO 94/21808, using the DBA/2 model and the Balb/c model as the mouse model.

### EXAMPLE 1

Comparative FACS Analysis of M-3 Cells Treated with Foreign Peptide by Various Methods

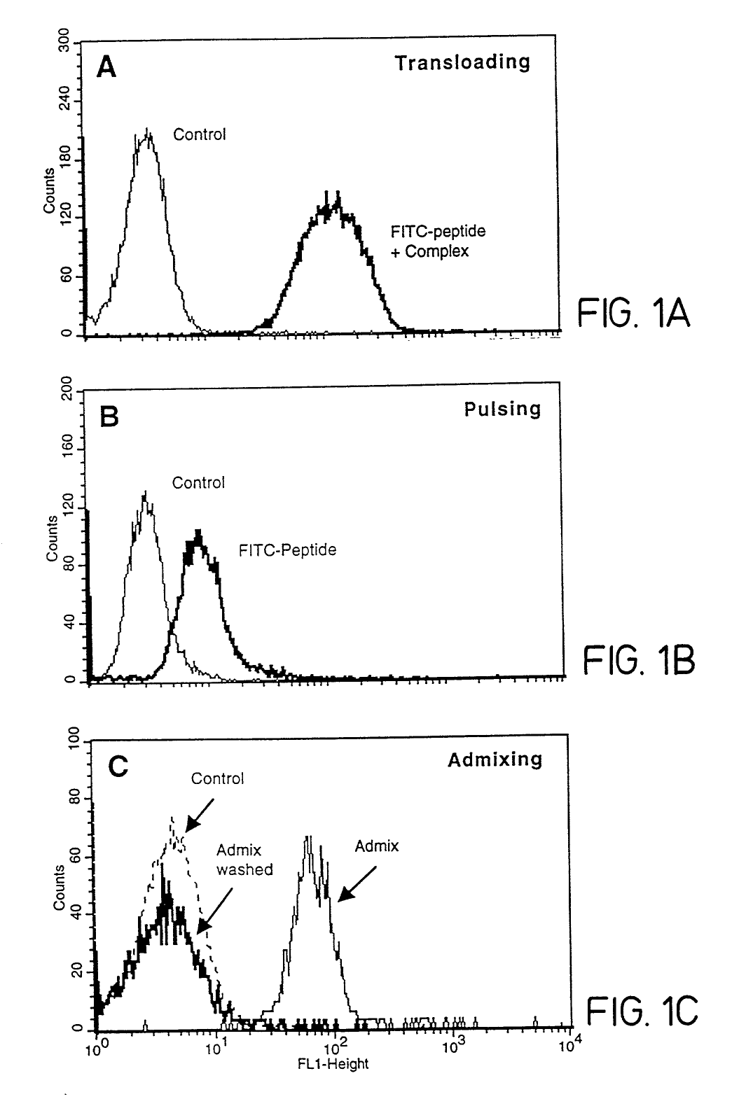

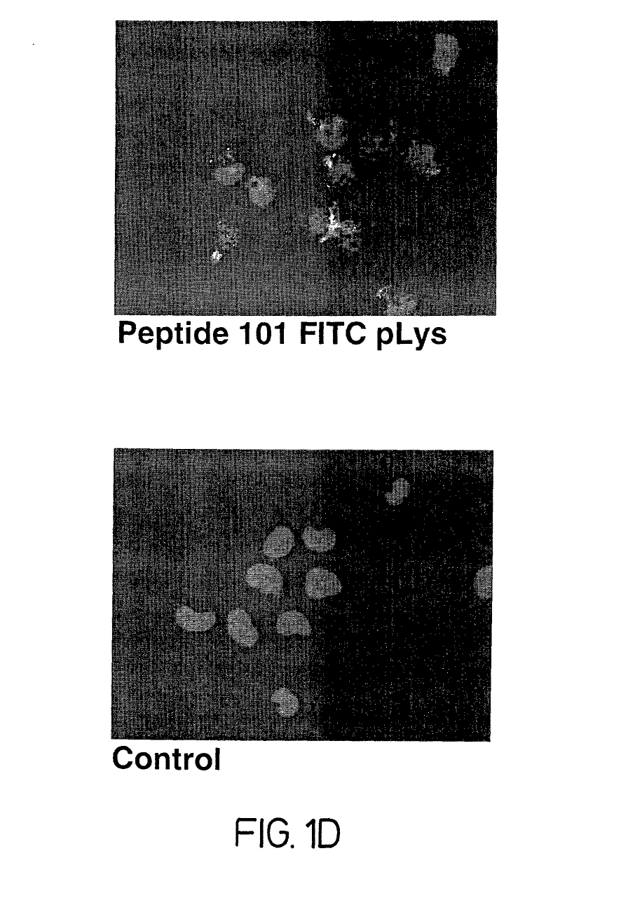

For this investigation, which is shown in FIG. 1, the xenopeptide LFEAIEGFI was applied to M-3 cells once with TfpL/DNA complexes (transloading; FIG. 1a), on another occasion the cells were incubated with the peptide (pulsing; FIG. 1b) and lastly the peptide was added as an adjuvant to the cells (FIG. 1c).

For the transloading, 160 μg of FITC-labelled xenopeptide LFEAIEGFI or unlabelled control peptide were mixed with 3 μg of transferrin-polylysine (TfpL), 10 μg of pL and 6 μg of psp65 (Boehringer Mannheim, LPS free) in 500 μl of HBS buffer. After 30 minutes at ambient temperature the above solution was added to a T 75 cell culture flask with 1.5×10⁶ M-3 cells in 20 ml of DMEM medium (10% FCS, 20 mM glucose) and incubated at 37° C. After 3 hours the cells were washed twice with PBS, detached using PBS/2 mM EDTA and resuspended in 1 ml of PBS/5% FCS for the FACS analysis.

The pulsing of the cells with the peptide was carried out using 1-2×10⁶ cells in 20 ml of DMEM with 450 μg of peptide (FITC labelled or unlabelled) for 3 hours at 37° C.

For the adjuvant mixing, before the FACS analysis, 10⁶ cells detached from the culture flask were incubated with 100 μg of FITC-labelled peptide in 1 ml of PBS/5% FCS for 30 minutes at ambient temperature. After the replacement of the PBS/5% FCS the cells were washed and analysed again. The FACS analysis was carried out in accordance with the manufacturer's instructions using an FACS vantage apparatus (Becton Dickinson), equipped with a 5 W Argon Laser, set to 100 mW at 488 nm. The results of the FACS analysis are shown in FIGS. 1a to 1c. FIG. 1d shows microphotographs of cytocentrifuged M-3 cells: the upper picture shows cells which had been given the peptide by means of the complex (transloading) whilst the bottom picture shows cells which had been incubated with the peptide (pulsing). DAPI was used for counterstaining the nucleus.

M-3 cells which had been charged with the complex containing the peptide showed a shift in fluorescence of nearly 2 powers of ten, compared with untreated cells or cells treated with polylysine alone, indicating efficient transfer of the peptide to the cells by means of TfpL/DNA complex (FIG. 1a). Incubation with peptide (pulsing) was less effective, as can be seen by the shift in fluorescence of only one power of ten, which was practically undetectable by fluorescent microscopy (FIG. 1d). In the case of the adjuvant mixing, the peptide disappeared after the washing step (FIG. 1c), which indicates that the peptide binding was at most negligible.

### EXAMPLE 2

Curing of DBA/2 Mice Having Melanoma Metastases, with a Vaccine of Foreign Peptide-charged Melanoma Cells (Therapeutic Mouse Model)

a) Preparation of a Tumour Vaccine from M-3 Cells

160 μg of Xenopeptide LFEAIEGFI were mixed with 3 μg of transferrin-polylysine (TfpL), 10 μg of pL and 6 μg of psp65 (LPS free) in 500 μl of HBS buffer. After 30 minutes at ambient temperature the above solution was added to a T 75 cell culture flask with 1.5×10⁶ M-3 cells in 20 ml of DMEM medium (10% FCS, 20 mM glucose) and incubated at 37° C. After 3 hours, the cells were mixed with 15 ml of fresh medium and incubated overnight at 37° C. with 5% CO₂. 4 hours before administration, the cells were irradiated with 20 Gy. The vaccine was prepared as described in WO 94/21808.

b) Effectiveness of the Tumour Vaccines

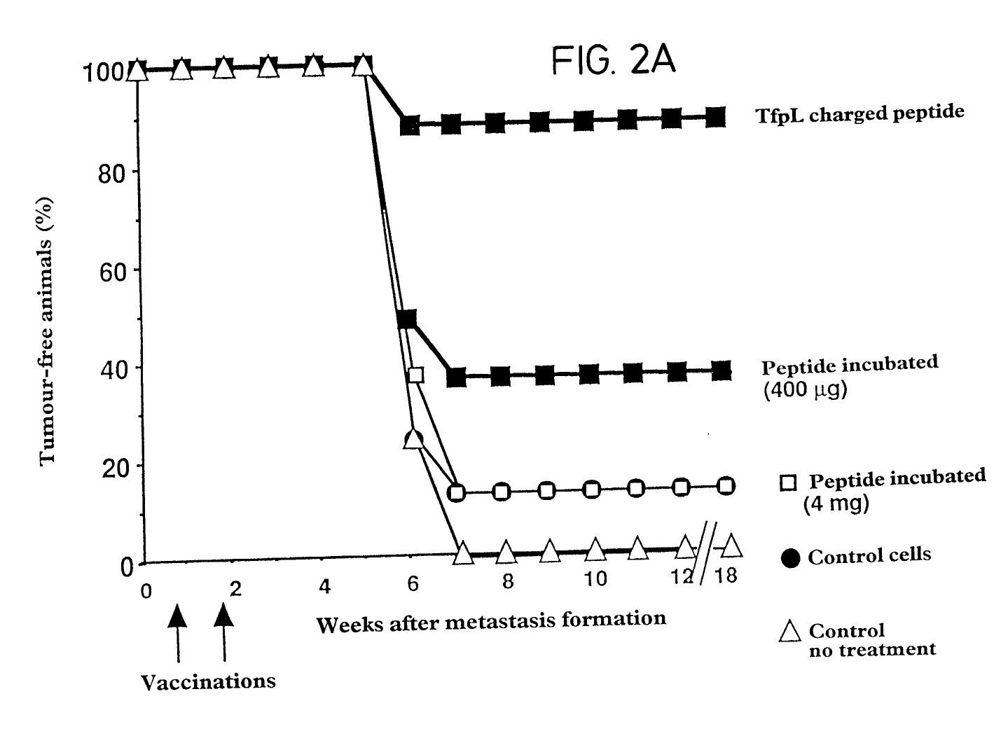

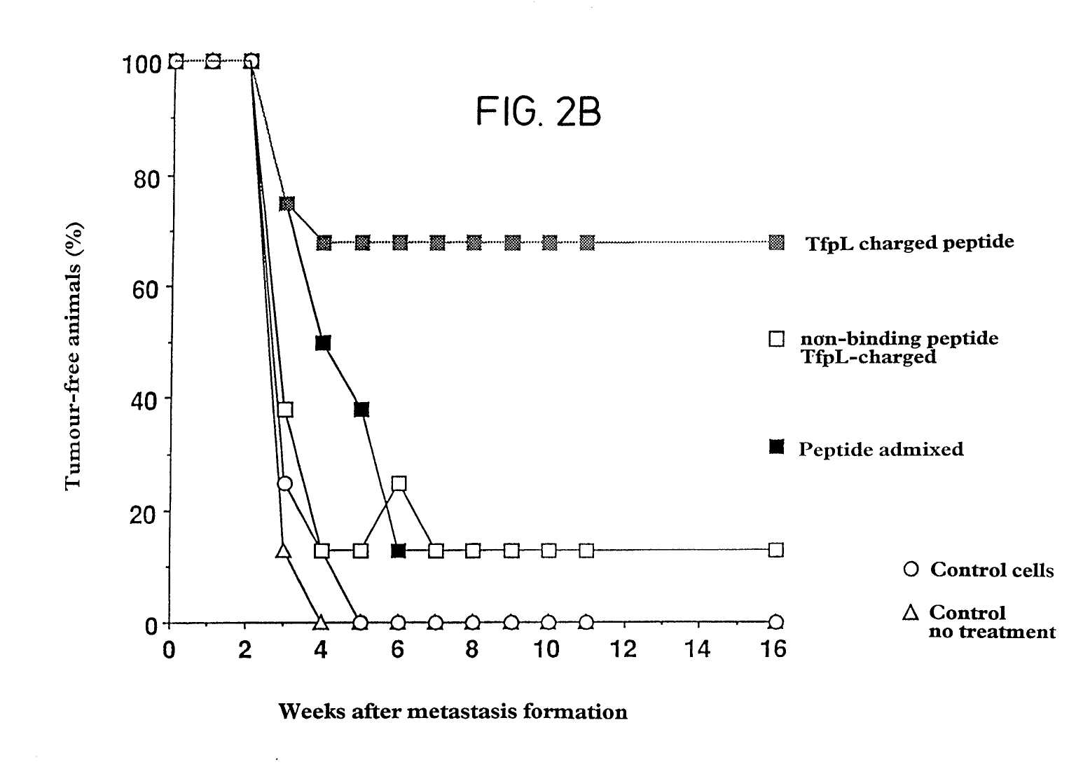

DBA/2 mice 6-12 weeks old with a 5 day metastasis (produced by the subcutaneous injection of 10⁴ live M-3 cells) were treated twice, at an interval of one week, by subcutaneous injection of the tumour vaccine (dose: 10⁵ cells/animal). There were 8 mice involved in the experiment. The results of the experiments are shown in FIG. 2a; it is apparent that 7 out of 8 animals were cured after the administration of the vaccine which contained peptide charged onto the tumour cells by means of TfpL/DNA complexes. In comparative tests, a vaccine was used in which the peptide LFEAIEGFI (400 μg or 4 mg) had been applied to the cells by incubation (3 hours at 37° C.; “pulsing”). Of the animals given a vaccine with 400 μg of peptide, 3 out of the 8 remained free from tumours; the vaccine consisting of cells treated with 4 mg of peptide cured only 1 out of 8 animals. Controls consisted of irradiated M-3 cells on their own and cells which had been charged with the complexes but without peptide (in each case 1/8 animals remained free from tumours). In the group of control animals which received no treatment of any kind, all the animals developed tumours.

In order to investigate the relevance, on the one hand, of the method of producing the vaccine and on the other hand the peptide sequence, another series of experiments was carried out; in these experiments, a highly tumorigenic variant of the M-3 cells was used. In the experiments in which the significance of the method of treatment was tested, vaccines were produced in which the peptide was not charged onto the cells using polylysine-transferrin but was merely mixed with the cells as an adjuvant. As a control for the peptide sequence, the anchor amino acids of the peptide at positions 2 and 9, namely phenylalanine and isoleucine, were replaced by proline and glycine, respectively, leading to the peptide Leu Pro Glu Ala Ile Glu Gly Phe Gly (LPEAIEGFG); this peptide lacks the ability to bind H2-K^{d}. Metastasis formation was monitored at least once a week. The results of these tests are shown in FIG. 2b. The vaccine, produced by charging the cells with LFEAIEGFI using the TfpL/DNA complexes, cured 6 out of 8 animals. On the other hand, 7 out of 8 animals given a vaccine for which the peptide LFEAIEGFI had simply been mixed with the cells or which consisted of cells which had been charged by means of TfpL/DNA complexes with the modified peptide LPEAIEGFG which did not bind to the HLA motif, developed tumours. In the control group, which had been treated only with irradiated M-3 cells or received no treatment at all, all the animals developed tumours.

c) Investigation of the Effect of the Quantity of Peptide in the Vaccine

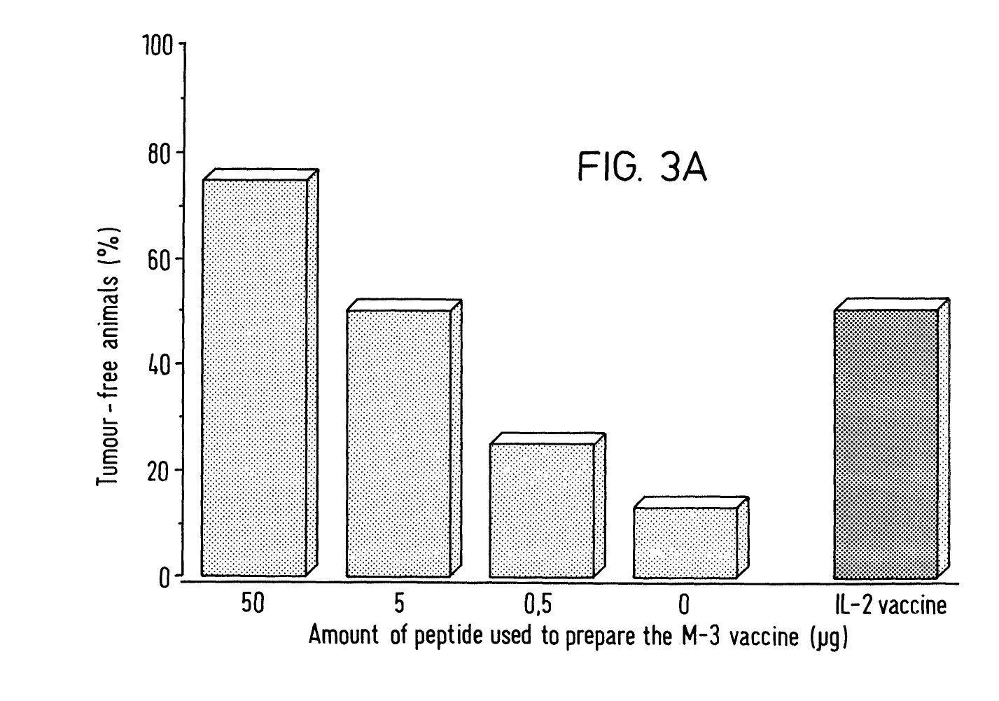

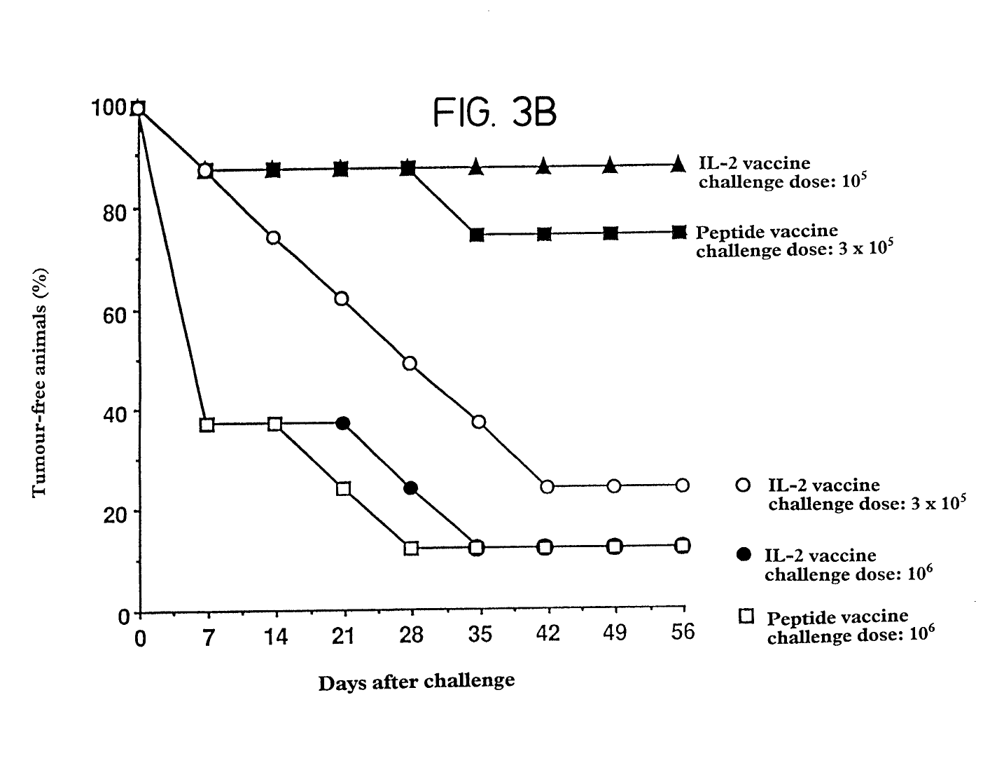

As described in a), peptide-containing complexes were prepared which contained either 50, 5 or 0.5 μg of the effective peptide LFEAIEGFI, and M-3 cells were charged therewith. An IL-2 vaccine which secreted the optimum dose of IL-2 (see d)) was used as a comparison. This vaccine was used to immunise DBA/2 mice which had a five-day metastasis. The vaccine containing 50 μg of peptide cured 6 out of 8 mice, the one containing 5 μg cured 4 out of 8 mice, like the IL-2 vaccine, whilst the vaccine containing 0.5 μg cured only 2 out of 8 animals. This experiment is shown in FIG. 3a.

### EXAMPLE 3

Comparison of the Vaccines Containing Foreign Peptide with a Tumour Vaccine from IL-2 Secreting Tumour Cells in the Prophylactic Mouse Model

In comparison tests, two groups of experimental animals (8 in each group) were pre-immunised twice, at intervals of one week, on the one hand with the vaccine described in EXAMPLE 2a) and on the other hand with a vaccine of IL-2 secreting M-3 cells (prepared in accordance with the procedure described in WO 94/21808, IL-2 dose 2,000 units per animal). One week after the last vaccination, contralateral tumours were set, with an increasing number of tumour cells (“challenge”; the dose is specified in FIG. 3b)). It was found that pre-immunisation with the tumour vaccine according to the invention was superior to treatment with the IL-2 vaccine: naive mice, vaccinated with the IL-2 vaccine, were protected only against a dose of 10⁵ live, highly tumorigenic cells (M-3-W). However, the capacity of this vaccine was exhausted by a challenge of 3×10⁵ cells, whereas a tumour load of this degree had been successfully overcome by animals pre-immunised with the vaccine of tumour cells charged with foreign peptide.

### EXAMPLE 4

Protection of Balb/c Mice by Pre-immunisation with a Vaccine of Foreign Peptide-charged Colon Carcinoma Cells (“Prophylactic Mouse Model”)

a) Preparation of the CT-26 Vaccine

160 μg of Xenopeptide LFEAIEGFI or FFIGALEEI were mixed with 12 μg of pL or with 3 μg of transferrin-polylysine plus 10 μg of polylysine and complexed for 30 minutes at ambient temperature in 500 μl of HBS buffer and then transferred into a T 75 cell culture flask with 1.5×10⁶ CT-26 cells in 4 ml of DMEM medium (10% FCS, 20 mM glucose), then incubated at 37° C. under 5% CO₂. After 4 hours, the cells were washed with PBS, mixed with 15 ml of fresh medium and incubated overnight at 37° C. under 5% CO₂. 4 hours before administration, the cells were irradiated with 100 Gy. The vaccine was prepared as described in WO 94/21808.

b) Testing the Effectiveness of the Cancer Vaccine for its Protective Effect Against CT-26 Challenge

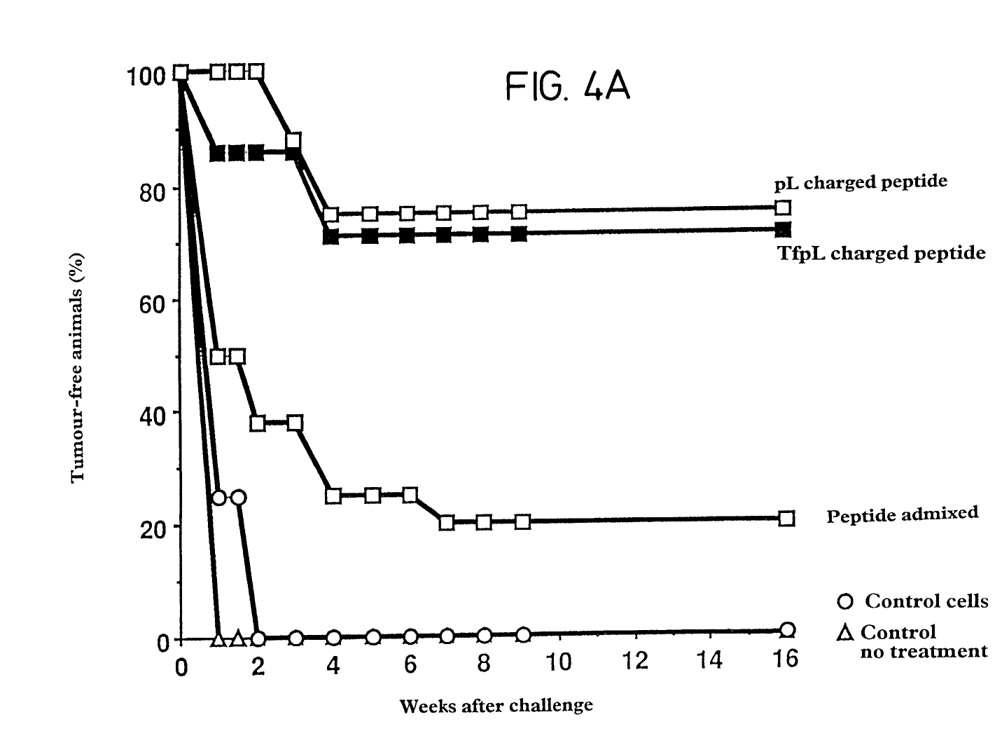

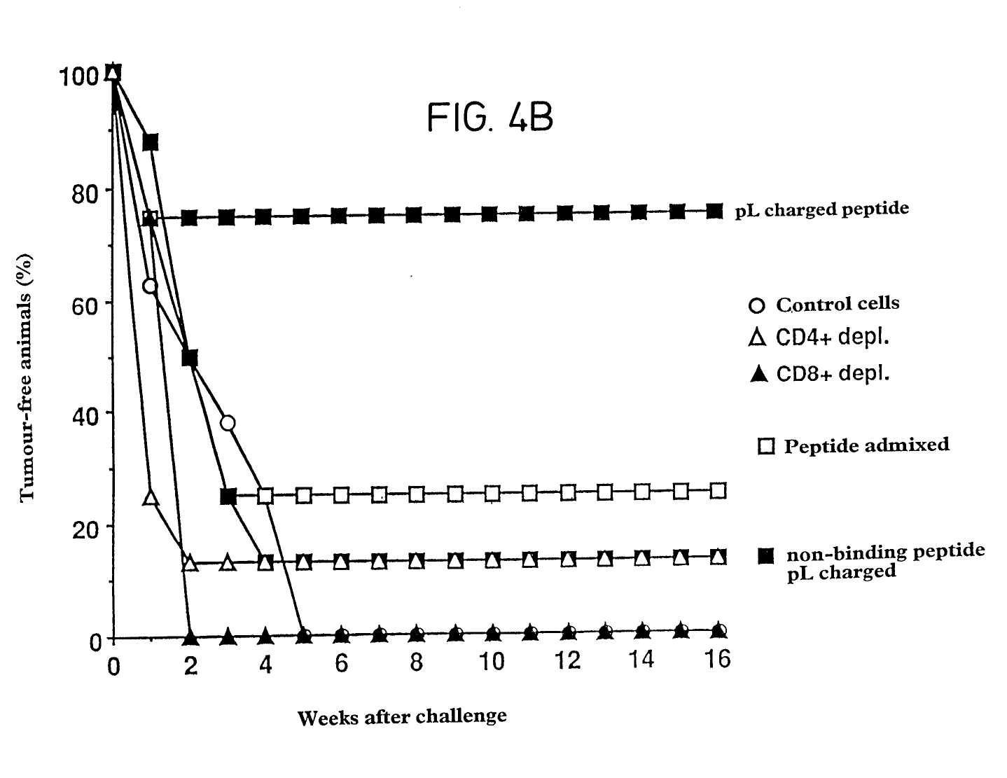

Balb/c mice 6-12 weeks old were vaccinated twice at an interval of one week by subcutaneous injection (cell dosage: 10⁵/mouse). There were 8 mice in each group (or 7 mice in the experiment in which pL was used to charge the cells) in the experiment. One week after the final vaccination, contralateral tumours were applied using 5×10⁴ parental CT-26 cells. Comparison tests in which the vaccine was prepared by a method other than using the complexes of TfpL/DNA, as well as the controls, were carried out as described in Example 2. The growth of the tumour challenge was checked at least once a week. The results for peptide LFEAIEGFI can be seen in FIG. 4a; 6 out of 8 animals were protected. In the case of peptide FFIGALEEI (not shown in FIG. 4a), 4 out of 8 animals were protected.

c) Participation of T-cells in the Activity of the Tumour Vaccine

In order to detect the participation of T-cells in the systemic immunity brought about by the CT-26 vaccine, in another experiment, 24 hours before vaccination, CD4⁺ cells were removed by intravenous injection of 500 μg of monoclonal antibody GK1.5 (ATCC TIB 207) and CD8⁺ cells were removed by intravenous injection of 500 μg of monoclonal antibody 2.43 (ATCC TIB 210). A positive control group was given the vaccine without the elimination of CD4⁺ cells and CD8⁺ cells. The results of the tests are shown in FIG. 4b. The participation of the T-cells is indicated by the fact that all the animals from whom the T-cells were removed developed tumours.

### EXAMPLE 5

Protection of C57BL/6J Mice by Pre-immunisation with a Vaccine of Melanoma Cells Charged with Foreign Peptide (“Prophylactic Mouse Model”)

In this Example, mice of the strain C57BL/6J were used as the experimental animals (with 8 animals in each group). The melanoma cells used were the B16-F10 cells (NIH DCT tumour depository; Fidler et al., 1975) which are syngenic for the mouse strain used.

The animals of all the experimental groups were vaccinated twice at an interval of one week by subcutaneous injection of 105 B16-F10 cells per mouse:

In one test series, the vaccine was produced by charging irradiated B16-F10 cells with the peptide of sequence ASNENMETM, as described in Example 2 for the vaccine from M-3 cells.

In parallel experiments, B16-F10 cells secreting IL-2 or GM-CSF (prepared by the procedure described in WO 94/21808) were used as the vaccine for pre-immunisation; the vaccine produced 1,000 units of IL-2 or 200 ng of GM-CSF per animal.

A control group received irradiated but otherwise untreated B16-F10 cells for the pre-immunisation.

One week after the last vaccination, tumours were set in the experimental animals using 1×10⁴ live, irradiated B16-F10 cells and the tumour growth was then monitored.

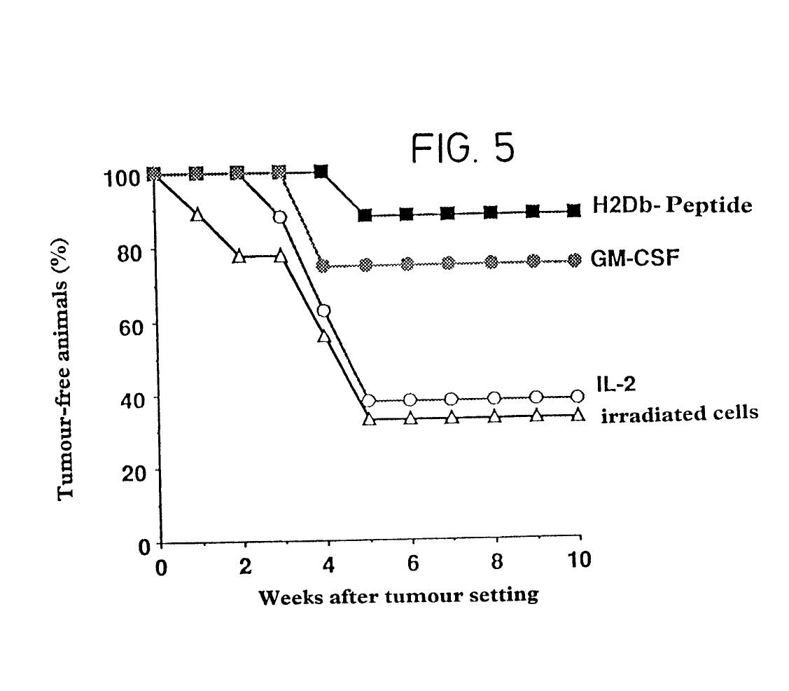

The results of these experiments are given in FIG. 5; the tumour cells charged with the foreign peptide exhibited the best protective effect against tumour formation.

TABLE

| | MHC | | |
|---|---|---|---|
| Peptide sequence | halotype | Antigen | Reference |
| SPSYVYHQF | L^{d} | gp70, | Huang and Pardoll, 1996 |
| | | endogenous | |
| | | MuLV | |
| FEQNTAQA | K^{b} | Connexin37 | Mandelboim, et al., 1994 |
| FEQNTAQP | K^{b} | Connexin37 | Mandelboim, et al., 1994 |
| SYFPEITHI | K^{d} | JAK1 | Rammensee, et al., 1995 |
| EADPTGHSY | HLA-A1 | MAGE-1 | Rammensee, et al., 1995 |
| EVDPIGHLY | HLA-A1 | MAGE-3 | Rammensee, et al., 1995 |
| YMNGTMSQV | HLA-A2 + | Tyrosinase | Rammensee, et al., 1995 |
| | HLA-A0201 | | |
| MLLALLYCL | HLA-A0201 | Tyrosinase | Rammensee, et al., 1995 |
| AAGIGILTV | HLA-A0201 | Melan A/ | Rammensee, et al., 1995 |
| | | Mart1 | |
| YLEPGPVTA | HLA-A0201 | pme117/ | Rammensee, et al., 1995 |
| | | gp100 | |
| ILDGTATLRL | HLA-A0201 | pme117/ | Rammensee, et al., 1995 |
| | | gp100 | |
| SYLDSGIHF | HLA-A24 | β-Catenin | Robbins, et al., 1996 |
| AINNYAQKL | D^{b} | SV-40 sized | Lill, et al., 1992 |
| CKGVNKEYL | | T-antigen | |
| QGINNLDNL | | | |
| NLDNLRDYL | | | |
| EEKLIVVLF | HLA-B44 | MUM-1 | Coulie, et al., 1995 |
| ACDPHSGHFV | HLA-A2 | mutated | Wolfel, et al., 1995 |
| | | CDK4 | |
| AYGLDFYIL | HLA-A24 | p15, | Robbins, et al., 1995 |
| | | unknown | |
| | | function | |
| KTWGQYWQV | HLA-A2 | gp100 | Kawakami and |
| YLEPGPVTA | | | Rosenberg, 1995 |
| HMTEVVRHC | HLA-A2 | mutated p53 | Houbiers, et al., 1993 |
| KYICNSSCM | K^{d} | mutated p53 | Noguchi, et al., 1994 |
| GLAPPQJJEI | HLA-A2 | mutated p53 | Stuber, et al., 1994 |
| LLGRNSEEM | | | |
| LLPENNVLSPL | HLA-A2 | wild-type | Theobald, et al., 1995 |
| | | p53 | |
| RMPEAAPPV | | | |
| LLGRNSFEV | | | |
| LLGRDSFEV | HLA-A2 | mutated p53 | Theobald, et al., 1995 |

### BIBLIOGRAPHY

Alexander, J. et al., 1989, Immunogenetics 29, 380

Allred, D. C. et al.,1992, J. Clin. Oncol. 10 (4), 599-605

Behr, J. P., 1994, Bioconjug-Chem., Sept-Oct, 5(5), 382-9

Biologic Therapy of Cancer, Editors: DeVita, V. T. Jr., Hellman, S., Rosenberg, S. A., Verlag J. B. Lippincott Company, Philadelphia, New York, London, Hagerstown

Boon, T., 1993, Spektrum der Wissenschaft (May), 58-66

Boon, T. et al., 1994, Annu. Rev. Immunol. 12, 337-65

Braciale, T. J. and Braciale, V. L., 1991, Immunol. Today 12, 124-129

Carrel, S. and Johnson, J. P., 1993, Current Opinion in Oncology 5, 383-389

Coligan, J. E., Kruisbeek, A. M., Margulies, D. H., Shevach, Falk, K. et al., 1991, Nature 351, 290-296

Coulie, P. G. et al., 1992, Int. J. Cancer, 50, 289-297

Coulie, P. G., Lehmann, F., Lethe, B., Herman, J., Lurquin, C., Andrawiss, M., and Boon, T. (1995). Proc Natl Acad Sci U S A 92, 7976-80

Cox, A. L. et al., 1994, Science 264, 5159, 716-9

Current Protocols in Molecular Biology, 1995, Publisher: Ausubel F. M., et al., John Wiley & Sons, Inc.

Dranoff, G. et al., 1993, Proc. Natl. Acad. Sci. USA 90, 3539-3543

Dranoff, G. and Mulligan, R. C., 1995, Advances in Immunology 58, 417

Falk, K. et al., 1991, Nature 351, 290-296

Felgner, J. H. et al., 1994, J. Biol. Chem. 269, 2550-2561

Fenton, R. G. et al., 1993, J. Natl. Cancer Inst. 85, 16, 1294-302

Fisk, B. et al., 1995, J. Exp. Med. 1881, 2109-2117

Flow Cytometry, Acad. Press, Methods in Cell Biology, 1989, Vol. 33, Publisher: Darzynkiewicz, Z. and Crissman, H. A.

Gedde Dahl, T. et al., 1992, Hum Immunol. 33, 4, 266-74

Guarini, A. et al., 1995, Cytokines and Molecular Therapy 1, 57-64

Han, X. K. et al., 1995, PNAS 92, 9747-9751

Handbook: FACS Vantage™ User's Guide, April 1994, Becton Dickinson

Handbook: CELL Quest™ Software User's Guide, June 1994, Becton Dickinson

Hérin M. et al., 1987, Int. J. Cancer, 39, 390

Hock, H. et al., 1993, Cancer Research 53, 714-716

Houbiers, J. G., Nijman, H. W., van der Burg, S. H., Drijfhout, J. W., Kenemans, P., van de Velde, C. J., Brand, A., Momburg, F., Kast, W. M., and Melief, C. J. (1993). Eur J Immunol 23, 2072-7.

Huang, A. Y. C., and Pardoll, D. M. (1996). Proc Natl Acad Sci U S A 93, 9730-5

Jung, S. et al., 1991, J. Exp. Med. 173, 1, 273-6

Kawakami, Y. et al., 1995, The Journal of Immunol. 154, 3961-3968

Kärre, K. et al., 1986, Nature 319, 20. Feb., 675

Kovacsovics Bankowski, M. and Rock, K. L., 1995, Science 267, 243-246

Lehmann, J. M. et al., 1989, Proc. Natl. Acad. Sci. USA 86, 9891-9895

Lethe, B. et al., 1992, Eur. J. Immunol. 22, 2283-2288

Lill, N. L., Tevethia, M. J., Hendrickson, W. G., and Tevethia, S. S. (1992). J Exp Med 176, 449-57

Loeffler, J.-P. et al., 1993, Methods Enzymol. 217, 599-618

Mackiewicz, A. et al., 1995, Human Gene Therapy 6, 805-811

Malnati, M. S. et al., 1995, Science 267, 1016-1018

Mandelboim, O. et al., 1994, Nature 369, 5.May, 67-71

Mandelboim, O. et al., 1995, Nature Medicine 1, 11, 1179-1183

Morishita, R. et al., 1993, J. Clin. Invest. 91, 6, 2580-5

Nabel, G. J. et al., 1993, Proc. Natl. Acad. Sci. USA 90, 11307-11311

Noguchi, Y., Chen, Y. T., and Old, L. J. (1994). Proc Natl Acad Sci U S A 91, 3171-3175

Oettgen, H. F. and Old, L. J., 1991, Biologic Therapy of Cancer, Editors: DeVita, V. T. Jr., Hellman, S., Rosenberg, S. A., Verlag J. B. Lippincott Company, Philadelphia, New York, London, Hagerstown, 87-119

Ostrand-Rosenberg, S., 1994, Current Opinion in Immunology 6, 722-727

Pardoll, D. M., 1993, Immunology Today 14, 6, 310

Practical Immunology, Editors: Leslie Hudson and Frank C. Hay, Blackwell Scientific Publications, Oxford, London, Edinburgh, Boston, Melbourne

Peace, D. J. et al., 1991, J. Immunol. 146, 6, 2059-65

Peoples, G. E. et al., 1994, J. Immunol. 152, 10, 4993-9

Plautz, G. E. et al., 1993, Proc. Natl. Acad. Sci. USA 90, 4645-4649

Rammensee, H. G. et al., 1993, Current Opinion in Immunology 5, 35-44

Rammensee, H. G., 1995, Current Opinion in Immunology 7, 85-96

Rammensee, H. G., Friede, T., and Stepvanovic, S. (1995). Immunogenetics 41, 178-228

Remy, J. S. et al., 1994, Bioconjug-Chem., Nov-Dec, 5(6), 647-54

Rivoltini, L. et al., 1995, The Journal of Immunology 154, 2257-2265

Robbins, P. F., el Gamil, M., Li, Y. F., Topalian, S. L., Rivoltini, L., Sakaguchi, K., Appella, E., Kawakami, Y., and Rosenberg, S. A. (1995). J Immunol 154, 5944-50

Robbins, and Rosenberg. (1996). J EXP MED 183, 1185-92.

Schmidt, W. et al., May 1995, Proc. Natl. Adac. Sci. USA, 92, 4711-4714

Skipper, J., and Stauss, H. J., 1993, J. Exp. Med. 177, 5, 1493-8

Slingluff, C. L. et al., 1994, Current Opinion in Immunology 6, 733-740

Stein, D. et al., 1994, EMBO-Journal, 13, 6, 1331-40

Stuber, G., Leder, G. H., Storkus, W. T., Lotze, M. T., Modrow, S., Szekely, L., Wolf, H., Klein, E., Karre, K., and Klein, G. (1994). Eur J Immunol 24, 765-768

Sykulev, Y. et al., 1994, Immunity 1, 15-22

Theobald, M., Levine, A. J., and Sherman, L. A. (1995) PNAS 92, 11993-7

Tibbets, L. M. et al., 1993, Cancer, Jan. 15., Vol.71, 2, 315-321

van der Bruggen, P. et al., 1994, Eur. J. Immunol. 24, 9, 2134-40 Issn: 0014-2980

Van Pel, A. and Boon, T., 1982, Proc. Natl. Acad. Sci. USA 79, 4718-4722

Wölfel, T. et al., 1994 a), Int. J. Cancer 57, 413-418

Wölfel, T. et al., 1994 b), Eur. J. Immunol. 24, 759-764

Wölfel, T., Hauer, M., Schneider, J., Serrano, M., Wolfel, C., Klehmann Hieb, E., De Plaen, E., Hankeln, T., Meyer zum Buschenfelde, K. H., and Beach, D. (1995). Science 269, 1281-4

York, I. A. and Rock, K. L., 1996, Ann. Rev. Immunol. 14, 369-296

Yoshino, I. et al., 1994 a), J. Immunol. 152, 5, 2393-400

Yoshino, I. et al., 1994 b), Cancer Res., 54, 13, 3387-90

Zatloukal, K. et al., 1993, Gene 135, 199-20

Zatloukal, K. et al., 1995, J. Immun. 154, 3406-3419

1. Tumour vaccine for administration to a patient, characterised in that it contains tumour cells which themselves present peptides derived from tumour antigens in an HLA context and at least some of which have at least one MHC-I-haplotype of the patient on the cell surface, and which have been charged with one or more peptides a) and/or b) in such a way that the tumour cells are recognised as foreign by the immune system of the patient, in context with the peptides, and trigger a cellular immune response, the peptides
a) acting as ligands for the MHC-I-haplotype which is common to the patient and the tumour cells of the vaccine, and are different from peptides which are derived from proteins expressed by the cells of the patient, or
b) acting as ligands for the MHC-I-haplotype, which is common to the patient and to the tumour cells of the vaccine, and are derived from tumour antigens which are expressed by the patient's cells and are present in a concentration on the tumour cells of the vaccine which is higher than the concentration of a peptide derived from the same tumour antigen as the one expressed on the patient's tumour cells.

2. Tumour vaccine according to claim 1, characterised in that it contains autologous tumour cells.

3. Tumour vaccine according to claim 1, characterised in that it contains allogenic tumour cells.

4. Tumour vaccine according to claim 3, characterised in that the allogenic tumour cells are cells of one or more cell lines, of which at least one cell line expresses at least one, preferably several tumour antigens, which are identical to the tumour antigens of the patient to be treated.

5. Tumour vaccine according to one of claims 1 to 4, characterised in that it consists of a mixture of autologous and allogenic cells.

6. Tumour vaccine according to claim 1, characterised in that peptide a) or b) is an H2-K^{d}-ligand.

7. Tumour vaccine according to claim 1, characterised in that peptide a) or b) is an H2-K^{b}-ligand.

8. Tumour vaccine according to claim 1, 6 or 7, characterised in that peptide a) is derived from a naturally occurring immunogenic protein or a cellular breakdown product thereof.

9. Tumour vaccine according to claim 8, characterised in that peptide a) is derived from a viral protein.

10. Tumour vaccine according to claim 9, characterised in that the peptide is derived from an influenza virus protein.

11. Tumour vaccine according to claim 10, characterised in that the peptide has the sequence Leu Phe Glu Ala Ile Glu Gly Phe Ile.

12. Tumour vaccine according to claim 10, characterised in that the peptide has the sequence Ala Ser Asn Glu Asn Met Glu Thr Met.

13. Tumour vaccine according to claim 8, characterised in that peptide a) is derived from a bacterial protein.

14. Tumour vaccine according to claim 1, characterised in that peptide a) is derived from a tumour antigen foreign to the patient.

15. Tumour vaccine according to claim 1, characterised in that peptide a) is a synthetic peptide.

16. Tumour vaccine according to claim 15, characterised in that the peptide has the sequence Phe Phe Ile Gly Ala Leu Glu Glu Ile.

17. Tumour vaccine according to one of claims 1 to 16, characterised in that the tumour cells have been treated with a number of peptides of different sequences.

18. Tumour vaccine according to claim 17, characterised in that the peptides differ in that they bind to different HLA-subtypes.

19. Tumour vaccine according to claim 17, characterised in that the peptides differ from one another in terms of their sequence which is not crucial to HLA-binding.

20. Tumour vaccine according to one of claims 1 to 19, characterised in that it also contains tumour cells which are transfected with a cytokine gene.

21. Tumour vaccine according to claim 20, characterised in that the cytokine is IL-2 and/or IFN-γ.

22. Tumour vaccine according to one of claims 1 to 21, characterised in that it also contains fibroblasts which have been treated with a peptide b).

23. Tumour vaccine according to one of claims 1 to 22, characterised in that it also contains dendritic cells which have been treated with a peptide b) and/or with a peptide binding to an MHC-II molecule.

24. Process for producing a tumour vaccine containing tumour cells for administering to a patient, characterised in that tumour cells which themselves present peptides derived from tumour antigens in an HLA context and of which at least some express at least one MHC-I-haplotype of the patient, are treated with one or more peptides which
a) act as ligands for the MHC-I-haplotype which is common to the patient and the tumour cells of the vaccine, and are different from peptides derived from proteins which are expressed by the patient's cells, or
b) act as ligands for the MHC-I-haplotype common to the patient and the tumour cells of the vaccine, and are derived from tumour antigens which are expressed by the patient's cells,
the tumour cells being incubated with one or more peptides a) and/or b) for such a time and in such a quantity, in the presence of an organic polycation, that the peptides are bound to the tumour cells in such a way that they are recognised as foreign by the patient's immune system, in context with the tumour cells, and trigger a cellular immune response.

25. Process according to claim 24, characterised in that polylysine is used as the polycation.

26. Process according to claim 25, characterised in that polylysine having a chain length of about 30 to about 300 lysine groups is used.

27. Process according to one of claims 24 to 26, characterised in that the polycation is used in an at least partially conjugated form.

28. Process according to claim 27, characterised in that the polycation is conjugated with transferrin.

29. Process according to one of claims 24 to 27, characterised in that the cells are also treated in the presence of DNA.

30. Process according to claim 29, characterised in that the DNA is a plasmid.

31. Process according to claim 29 or 30, characterised in that the ratio of DNA to polycation, which is optionally partially conjugated with a protein, is about 1:2 to about 1:5.

32. Process according to one of claims 29 to 31, characterised in that the cells are melanoma cells.

33. Process according to claim 24, characterised in that peptide a) and/or b) is used in an amount of about 50 μg to about 160 μg per 1×10⁵ to 2×10⁷ cells.

34. Application of the process according to one of claims 24 to 32 to fibroblasts, in which a peptide b) derived from a tumour antigen of the patient is used as the peptide.

35. Application of the process according to one of claims 24 to 33 to dendritic cells, in which a peptide b) derived from a tumour antigen of the patient and/or a peptide which binds to an MHC-II molecule of the patient is used as the peptide.
